(19) United States
(12) Patent Application Publication
(10) Pub. No.: US 2017/0358232 A1
(43) Pub. Date: Dec. 14, 2017

(54) STUDENT ENGAGEMENT SYSTEM, DEVICE, AND METHOD

(71) Applicant: Victoria L. Adams, Hanoverton, OH (US)

(72) Inventor: Victoria L. Adams, Hanoverton, OH (US)

(21) Appl. No.: 15/615,040

(22) Filed: Jun. 6, 2017

Related U.S. Application Data
(60) Provisional application No. 62/347,315, filed on Jun. 8, 2016.

(51) Int. Cl. G09B 5/14; G06F 3/01; A61B 5/16; G06F 19/00; H04L 29/06; G06F 1/16

(52) U.S. Cl. CPC G09B 5/14; H04L 63/08; G06F 1/163; A61B 5/165; G06F 19/3406; G06F 1/1698; G06F 3/016

(57) ABSTRACT

A student engagement device, system and method is provided that encourages students to maintain student engagement during a classroom activity or lecture. The device may be worn by the student and include a display that displays signals received from a remote controller. The signals are generated in the remote control based on subjective observation from the teacher related to student to engagement and may be irrespective of student behavior. The system may further include a computer device accessible by the student or parent for viewing student engagement levels during a class or over a longer length of time, such as a week, month, semester, or year.

### CROSS REFERENCE TO RELATED APPLICATIONS

This application claims the benefit of U.S. Provisional Application Ser. No. 62/347,315, filed on Jun. 8, 2016; the entirety of which is incorporated herein by reference as if fully rewritten.

### BACKGROUND

### Technical Field

The present disclosure relates to systems, devices, and methods associated with teaching students. More particularly, the present disclosure relates to engaging students to foster learning. Specifically, the present disclosure relates to an engagement system utilizing a wearable electronic device presenting student engagement information to the student input from the teacher, which may differ from student behavior.

### Background Information

The relationship between students and a teacher has existed for quite some time and will continue to transcend generations to come. In this relationship, typically the teacher has information that is being conveyed to the student so the student can learn the information. In order for the student to learn, typically they must be engaged. Student engagement refers to the degree of attention, curiosity, interest, optimism, and passion that students show when they are learning or being taught, which extends to the level of motivation they have to learn and progress in their education.

Others have attempted to utilize various devices to encourage and track student engagement. These are shown in: U.S. Pat. Publ. 2001/0029319; U.S. Pat. Publ. 2003/0030544; U.S. Pat. Publ. 2008/0227065; U.S. Pat. Publ. 2011/0128151; and U.S. Pat. Publ. 2012/0244503.

U.S. Pat. Publ. 2001/0029319 (the '319 disclosure) discloses a system and method for monitoring the activity level of one or more individuals and modifying the behavior of those individuals based on feedback from the activity level monitor. When applying the system and method to hyperactive children in a classroom environment, an activity monitor is attached to each hyperactive child. The activity monitor determines the intensity of the subject's activity at the end of each epoch (approximately every 5 seconds), stores the determined intensity, and compares the determined intensity to an epoch threshold. If the determined intensity exceeds the epoch threshold, the hyperactive child is given vibrotactile feedback by the attached activity monitor. The length of time that the vibrotactile feedback is applied is proportional to the amount the determined epoch intensity exceeds the epoch threshold. When the hyperactive child presses a button/key on the activity monitor to thereby request session feedback, the session intensity is compared to two different session thresholds, and one of three LEDs on the activity monitor is lit up, depending on where the session intensity is in comparison to the two session thresholds. A base station, either a simple hand-held device or a more complicated desk-top device, is under the control of the teacher and has a wireless communication link with the activity monitors so that information may be downloaded and the activity monitors may be controlled. The system and method monitor the intensity of the movements of the hyperactive child, as well as monitoring the level of physical movement, which is merely the number of movements per unit time. Further, the system and method should record how the intensity of the physical movements changes over time so that a more detailed analysis of the child's activity may be made. The '319 disclosure focuses on tracking physical activity levels of the student, which the '319 disclosure believes is indicative of engagement, in order to monitor/change said behavior (i.e., try to get the student to calm down and stay more still). The '319 disclosure does not visually show a student how engaged they are in his or her learning based on direct input from the teacher in the classroom, who is in the best position to indicate the engagement of the student. As is well known, student engagement is different than behavior.

U.S. Pat. Publ. 2003/0030544 (the '544 disclosure) provides a remotely activated sensory stimulation device comprised of a case, a tactile stimulatory means, a receiving unit, a transmitting unit and a controller. The stimulation device is activated by remote control by a teacher or therapist attempting to teach a particular behavioral pattern. The amplitude and frequency of the tactile stimulation is adjustable to provide different prompts or to alter the prompt. The device is further camouflaged with a second functionality to eliminate any social stigma. The device is useful for teaching individuals with a variety of learning disorders including Autistic Spectrum Disorder, Attention Deficit Disorder, and Attention Deficit Hyperactivity Disorder. Essentially, the '544 disclosure is a device for training to decrease or eliminate an undesirable behavior through sensory signals. The intensity and frequency were able to be adjusted for individual students. While the '544 disclosure does indicate that the teacher can control the stimulation device, the '544 disclosure focuses on tactile indicators to monitor and change said behavior. The '544 disclosure does not visually show a student how engaged they are in the learning process based on direct input from the teacher in the classroom, who is in the best position to indicate the engagement of the student. In fact, the '544 disclosure indicates the opposite by stating that visual cues are insufficient for children with Spectrum Disorders.

U.S. Pat. Publ. 2008/0227065 (the '065 disclosure) provides an apparatus and method for enabling communication between an administrator and a student regarding behavior activities of the student. The apparatus comprises (a) a transmitter configured to be carried by an administrator, and (b) a receiver configured to be carried by a student. The transmitter and receiver are configured to (i) be selectively placed in circuit communication with each other by an initiation signal originating from the transmitter or receiver, (ii) enable communication from the administrator to the student via the receiver regarding behavioral activities of the student or communication from the student to the administrator if the student feels a need to communicate with the administrator regarding behavioral activities of the student, and (iii) enable the receiver to communicate with the student in a predetermined fashion regarding behavior activities of the student in the absence of communications from the administrator. This apparatus and method utilizes “credits” awarded to students based on teacher's cues. This device is described as being a mode of communication between a teacher and student (or adult and child). The device includes a teacher transmitter and a student wrist band. The devices store data regarding communication and can be uploaded to a software system for analysis. The software system allows for users to first gain an understanding of how to use the devices and later be able to analyze use over a long period of time. The system is based on the student receiving credit reward points for behaving in class. At the start of a class, a student accumulates credits for behaving for set intervals of time (predetermined and adjusted by the teacher using software). The teacher cues the student if they are not acting (i.e., behaving) appropriately and the student receives a signal from the receiver. The signals can be auditory, kinesthetic (vibrate), or visual. If the student continues misbehaving, credits are lost. The display on the wristband allows for the student to see how many credits they have and the duration of the intervals. Thus, the '065 disclosure focuses on tracking behavior to monitor/change said behavior and does not visually show a student how engaged they are in his or her learning. As is well known, student engagement is different than student behavior.

U.S. Pat. Publ. 2011/0128151 (the '151 disclosure) provides an attention assistance device for providing attention assistance including various features that help maintain a user's focus on a given task. The attention assistance device may include an activity sensor that generates an activity output in response to detection of a user generated activity. The attention assistance device may also have a timer that delineates increments in a counter at regular time intervals, resets the counter in response to the activity output, and generates a timer output when the counter reaches a threshold. Further, the attention assistance device may include a stimulation unit that alerts a user in response to the timer output. This device is used specifically for individuals with attention disorders. It is used as a memory device to maintain focus on a writing task. The device can take several forms, including a pen or watch. There are several sensors that correspond to a pen type device touching a surface. The device has a timing mechanism that can be set for intervals. If no movement is signaled after a given period, a stimulator is activated, such as a light, pulse, or sound. The device is linked to a computer system where information is stored, including amount of times and types of stimulation used. The device has a sleep mode and also display screen with an on/off switch. Essentially, this device monitors student attention based on movement. Thus, the '151 disclosure focuses on tracking behavior to monitor/change said behavior and does not visually show a student how engaged he or she is in their learning.

U.S. Pat. Publ. 2012/0244503 (the '503 disclosure) provides a method of attention recovery and behavioral modification comprising a wearable device that alerts a user over provided intervals to facilitate re-engagement and regained focus on a particular activity. The method involves utilizing a vibratory or similar sensory stimulation means from a wristband that is adapted to train a user to maintain focus or easily regain focus if the user's mind has begun to wander. The wristband is adapted to alert a user at defined intervals over a period of use or on demand remotely, which reminds the user that he or she should re-engage in an activity (meeting, classroom, etc.), wherein the method trains the user over time to regain focus on his or her own based on expected stimulus from the band that may be removed after extended exposure thereto. The method also contemplates tracking attention spans and success rates, along with reward systems for improving users. Essentially, this device utilizes regular intervals to alert the person to stay focused. Thus, the '503 disclosure focuses on tracking behavior to monitor/change said behavior and does not visually show a student how engaged he or she is in their learning.

### SUMMARY

Most of the prior art focuses on the teacher prompting a student to work and focuses on individuals with attention disorders in order to change his or her behavior. Again, the focus of the prior art is on behavior. However, student behavior is different than student engagement. The present disclosure puts forth the notion that prompting of a student from a teacher to correct his or her behavior may not be most efficient method for a student to learn. Particularly, there has been a fundamental shift in education in the last decade. The shift in education theory removed the former “drill and kill” mentality with facts and skills needing to be remembered and rendered in an assignment to now wanting students to demonstrate critical thinking skills in all types of situations. Teachers want students to lead their learning with questioning, exploring, discussing, and discovering. Teachers want students to be actively engaged in their learning, not just to complete a task at a given time. Activities are no longer completed with paper and pencil, but vary dependent on the students' needs and often utilize technology and other stimulating modes that students are interested. One stimulating mode often utilized by a teacher is a formal lecture. The lecture part is when students most often allow themselves to be passive and do not realize they are not actively taking part. As such, issues continue to exists with current student engagement systems, devices, and methods thereof, especially during a lecture period.

In accordance with the present disclosure, one embodiment of a student engagement system, device, and method may provide a mechanism to visually show a student how engaged he or she is in his or her learning (i.e., identify their student engagement; not necessarily their behavior). The system of the present disclosure may not focus on completing a task, nor changing/stopping a behavior as is prevalent in the prior art. Rather, the teacher is entering and sending feedback on positive student engagement instead of commands or signals to get back to work or pay attention. By the teacher focusing on positive student engagement (and thus not focusing on negative behaviors), the system fosters ownership of student learning.

In accordance with the present disclosure, one embodiment of a student engagement system, device, and method identifies and takes advantage of differences in situations that are employed and takes advantage of differences in situations that are intent-quantitative vs. qualitative circumstances. Such as when a system prompts the student to work to get a task done versus when the system provides positive feedback on student engagement to participate and ultimately learn more on their own, instead of corrective cues. The present system enables the student to make the active choice to learn and acknowledge when and how to become more involved in the learning process instead of dictating the learning, which is common to the prior art.

In accordance with the present disclosure, one embodiment of a student engagement system, device, and method facilitates learning in situations that are not task oriented, rather situations that are lecture based. These are the times that even the most agreeable and focused student may lack the self-monitoring skills to know if he or she is engaged in the lesson or not. All students can tell if they are completing a task or not regardless of if they struggle to stay on task and complete it in a given time frame. Yet, a larger population struggles with being able to judge if they “paid attention” or not. Being engaged is not just listening but asking questions and providing relevant discussion. Furthermore, in one particular embodiment, the device may not measure movement. Movement, even writing, is not indicative of purposeful learning or achievement.

In accordance with the present disclosure, one embodiment of a student engagement system, device, and method enables the teacher to only cue positive student engagement as opposed to alerting the negative behavior shown in the prior art. The present disclosure device displays and provides ongoing visual monitoring by the teacher who indicates positive student engagement. Typical negative behavior associated with not paying attention, acting inappropriately, or being off task is only recognized by the device to the extent that there is a decreasing display indicator.

In accordance with the present disclosure, one embodiment may provide a student engagement system comprising: a teacher; a plurality of students; a plurality of wearable electronic devices including wireless communication logic, wherein one student wears one wearable electronic device; a remote control operated by the teacher, wherein the remote control includes teacher-selected input cues configured to identify student engagement; one or more computing devices hosting a student portal associated with one of the plurality of students, wherein the computing device includes wireless communication logic; and a wireless network linking the plurality of wearable electronic devices, the remote control and the one or more computing devices; wherein the teacher enters and sends feedback from the remote to the wearable electronic device of positive engaged behavior to indicate to each student his or her level of engagement to foster student learning.

In yet another aspect, an exemplary embodiment of the present disclosure may provide a method of use from an exemplary school board's perspective comprising: providing a plurality of wearable student engagement monitors including a display; providing a first wearable student engagement monitor to a first student; providing a second wearable student engagement monitor to a second student; providing a controller to a teacher, wherein the controller is in wireless communication with the plurality of wearable student engagement monitors; establishing, via teacher input into the controller, a first engagement period and a second engagement period; illuminating the display during the first engagement period; requiring the teacher to indicate whether positive student engagement was present for each student during the first engagement period; wherein if the teacher indicates that student engagement was sufficient during the first engagement period, then the display remains fully illuminated during the second engagement period; wherein if the teacher indicates that student engagement was insufficient during the first engagement period, then the display is partially illuminated during the second engagement period or illuminated in a different manner so as to indicate a lapse in student engagement to the student; and wherein student engagement is independent and distinct from student behavior, and student engagement includes a degree of attention, curiosity, interest, optimism, and passion that students show when learning or being taught.

In yet another aspect, an exemplary embodiment of the present disclosure may provide a method of use from an exemplary teacher's perspective comprising: equipping students in a classroom with a wearable device including a display and wireless communication logic; generating a first engagement time period and a second engagement time period; teaching a classroom activity; engaging with the students and observing student engagement irrespective of behavior; generating a signal, via teacher input, in a remote controller; sending the signal to each wearable device, wherein if the student was engaged during the first time period, then the signal instructs the device to illuminate a first amount of lights during the second time period, and if the student was not engaged during the first time period, based on teacher observation, then the signal instructs the device to illuminate less than the first amount of lights during the second time period or the same amount of lights in a different color so as to indicate non-engagement to the student. Again, in this scenario, student engagement may be independent and distinct from student behavior, and student engagement includes a degree of attention, curiosity, interest, optimism, and passion that students show when learning or being taught.

In yet another aspect, an exemplary embodiment of the present disclosure may provide a student engagement method comprising: receiving a wireless signal carrying instructions for execution by processors housed in a device worn by a student in a classroom; executing the instructions via processors in the device worn by the student, wherein the instructions include operations to illuminate a light carried by the device when a wearer of the device is engaged in learning irrespective of student behavior, and wherein the signal is generated, via teacher input, in a remote controller based on student engagement during a classroom activity and student engagement is determined subjectively by a teacher.

In yet another aspect, an exemplary embodiment of the present disclosure may provide a student engagement method comprising: receiving a wireless signal carrying instructions for execution by processors housed in a device worn by a student in a classroom; executing the instructions via processors in the device worn by the student, wherein the instructions include operations to notify the student wearing the device of his or her student engagement in learning irrespective of student behavior; and wherein the signal is generated, via teacher input, in a remote controller based on student engagement during a classroom activity and student engagement is determined subjectively by a teacher. This exemplary method or another exemplary embodiment may further include receiving a plurality of time frames from the remote controller operated by the teacher, wherein the plurality of time frames includes a first engagement period and a second engagement period. This exemplary method or another exemplary embodiment may further include determining whether a first signal of positive student engagement has been received during the first engagement period; if the first signal has been received, then maintaining illumination of a light during the second engagement period, and if no first signal is received then ceasing illumination of the light during the second engagement period until the first signal is received, and then re-illuminating the light after the first signal is received. This exemplary method or another exemplary embodiment may further include processing the instructions in the device worn by the student; activating at least one light based on the instructions, wherein the light represents a level of student engagement subjectively determined through observation by the teacher; adjusting the illumination of the light in a display of the device after receiving a second signal indicating student engagement has returned after a period of non-engagement; and interrupting a period in which the student registers as non-engaged with a first engagement signal generated by the remote controller and identifying the student as engaged so as to illuminate the display in a manner indicating engagement within the same period. This exemplary method or another exemplary embodiment may further include aggregating the levels of student engagement during the classroom activity and sending the levels of student engagement to a database accessible at a later time via a computer application; collating and comparing levels of student engagement over multiple time periods, and providing a system recommendation of student engagement based on the collated and compared levels of student engagement; grouping periods of engaged activity to form engagement blocks viewable in the computer application, and grouping periods of non-engaged activity to form non-engagement blocks viewable in the computer application. This exemplary method or another exemplary embodiment may further include relaying the first engagement signal regarding student engagement from the wearable device to the database so as to enable the viewing of the teacher-generated signals via the computer application. This exemplary method or another exemplary embodiment may further include discretely notifying the student of non-engagement so as to not cause distractions or draw attention of other students to the non-engaged student. This exemplary method or another exemplary embodiment may further include actively observing for signals generated by the remote controller. This exemplary method or another exemplary embodiment may further include passively observing for signals generated by the remote controller. This exemplary method or another exemplary embodiment may further include wherein student engagement is independent and distinct from student behavior, and student engagement consists of a degree of attention, curiosity, interest, optimism, and passion shown by the student when learning or being taught.

In yet another aspect, an exemplary embodiment of the present disclosure may provide a wearable device comprising: a display carried by a housing sized to be generally wrist-worn adapted to display a level of student engagement; at least one non-transitory computer readable storage medium having instructions encoded thereon that when executed by one or more processors carried by the housing perform operations to display the level of student engagement in the display, the operations including: (i) receive a plurality of time frames including a first engagement period and a second engagement period from a distinct and remote controller generated via teacher input; (ii) receive signals generated via teacher input from the remote controller, wherein the signals are based on student engagement observed by the teacher during the first engagement period, and the signals represent positive feedback and no signals are sent representing inappropriate behavior; and (iii) notify the student by altering the display to represent a decline in student engagement in the event the teacher has not observed sufficient student engagement during the engagement period and has not generated the signal representing positive student engagement; and wherein student engagement is independent and distinct from student behavior, and student engagement includes a degree of attention, curiosity, interest, optimism, and passion shown by a student when learning or being taught. This exemplary device or another exemplary device may further include wherein the student engagement consists of only the degree of attention, curiosity, interest, optimism, and passion shown by the student when learning or being taught and does not include student behavior. This exemplary device or another exemplary device may further include at least one light in the display that illuminates in response to signals generated from a remote controller, wherein the light remains illuminated during the engagement period and either darkens or changes color after the illumination period if no signal representing positive student engagement was received by the wearable device from the remote controller operated by the teacher. This exemplary device or another exemplary device may further include wherein the at least one light is one of three light emitting diodes (LEDs); wherein the engagement period is one of three engagement periods; and wherein each one of the LEDs is associated with one engagement period. This exemplary device or another exemplary device may further include a receiver carried by the housing to received signals from the remote controller, wherein if the receiver receives a positive student engagement signal from the controller during a first engagement period, then all three LEDs remain illuminated in a first color, and if the receiver does not receive the positive student engagement signal from the controller during the first engagement period, then one of the LEDs will illuminate in a second color until the receiver receives the positive student engagement signal. This exemplary device or another exemplary device may further include wherein each one of the three engagement periods is in a range from five minutes to fifteen minutes. Alternatively, the three engagement periods may be in a range from about two minutes to about eight minutes. This exemplary device or another exemplary device may further include communication logic electrically connected with the at least one non-transitory computer readable storage medium effectuating wireless data transfer between the wearable device and a central server hosting an application displaying results of the student's engagement to a third party. This exemplary device or another exemplary device may further include timing logic in operative communication with the at least one non-transitory computer readable storage medium having instructions encoded thereon configured to initiate a countdown timer during which the student is to maintain his or her student engagement.

In yet another aspect, an embodiment of the present disclosure may provide a student engagement system comprising: a plurality of wearable electronic devices including wireless communication logic, wherein one student wears one wearable electronic device; a remote control operated by the teacher, wherein the remote control includes teacher-selected input cues configured to identify student engagement; one or more computing devices hosting a student portal associated with one of the plurality of students, wherein the computing device includes wireless communication logic; a wireless network linking the plurality of wearable electronic devices, the remote control and the one or more computing devices; wherein the teacher enters and sends signals generated by subjective feedback of student engagement from the remote to the wearable electronic device to indicated to each student his or her level of student engagement to foster student learning; and wherein student engagement is independent and distinct from student behavior, and student engagement includes a degree of attention, curiosity, interest, optimism, and passion shown by a student when learning or being taught.

In yet another aspect, an embodiment of the present disclosure may provide a student engagement device, system and method that encourages students to maintain student engagement during a classroom activity or lecture. The device may be worn by the student and include a display that displays signals received from a remote controller. The signals are generated in the remote control based on subjective observation from the teacher related to student to engagement and may be irrespective of student behavior. The system may further include a computer device accessible by the student or parent for viewing student engagement levels during a class or over a longer length of time, such as a week, month, semester, or year.

### BRIEF DESCRIPTION OF THE SEVERAL VIEWS OF THE DRAWINGS

A sample embodiment of the disclosure is set forth in the following description, is shown in the drawings and is particularly and distinctly pointed out and set forth in the appended claims. The accompanying drawings, which are fully incorporated herein and constitute a part of the specification, illustrate various examples, methods, and other example embodiments of various aspects of the disclosure. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. In some examples, an element shown as an internal component of another element may be implemented as an external component and vice versa. Furthermore, elements may not be drawn to scale.

FIG. 1 (FIG. 1) is a schematic view of a student engagement system in accordance with the present disclosure.

FIG. 2 (FIG. 2) is a schematic top view of an exemplary classroom setting in which the student engagement system operates.

FIG. 3 (FIG. 3) is a top view of a wearable electronic device for the student engagement system worn on the wrist of a user.

FIG. 3A (FIG. 3A) is an enlarged top view of the display on the wearable electronic device indicating full engagement of the student.

FIG. 3B (FIG. 3B) is an enlarged top view of the display on the wearable electronic device indicating a first non-engagement period of the student.

FIG. 3C (FIG. 3C) is an enlarged top view of the display on the wearable electronic device indicating a second non-engagement period of the student.

FIG. 4 (FIG. 4) is a schematic view of a computing environment in which portions of the student engagement system operate.

FIG. 5 (FIG. 5) is a schematic view of a computing device or tablet hosting a student portal of the student engagement system.

FIG. 6 (FIG. 6) is a flow chart representing a method in accordance with the present disclosure.

Similar numbers refer to similar parts throughout the drawings.

### DETAILED DESCRIPTION

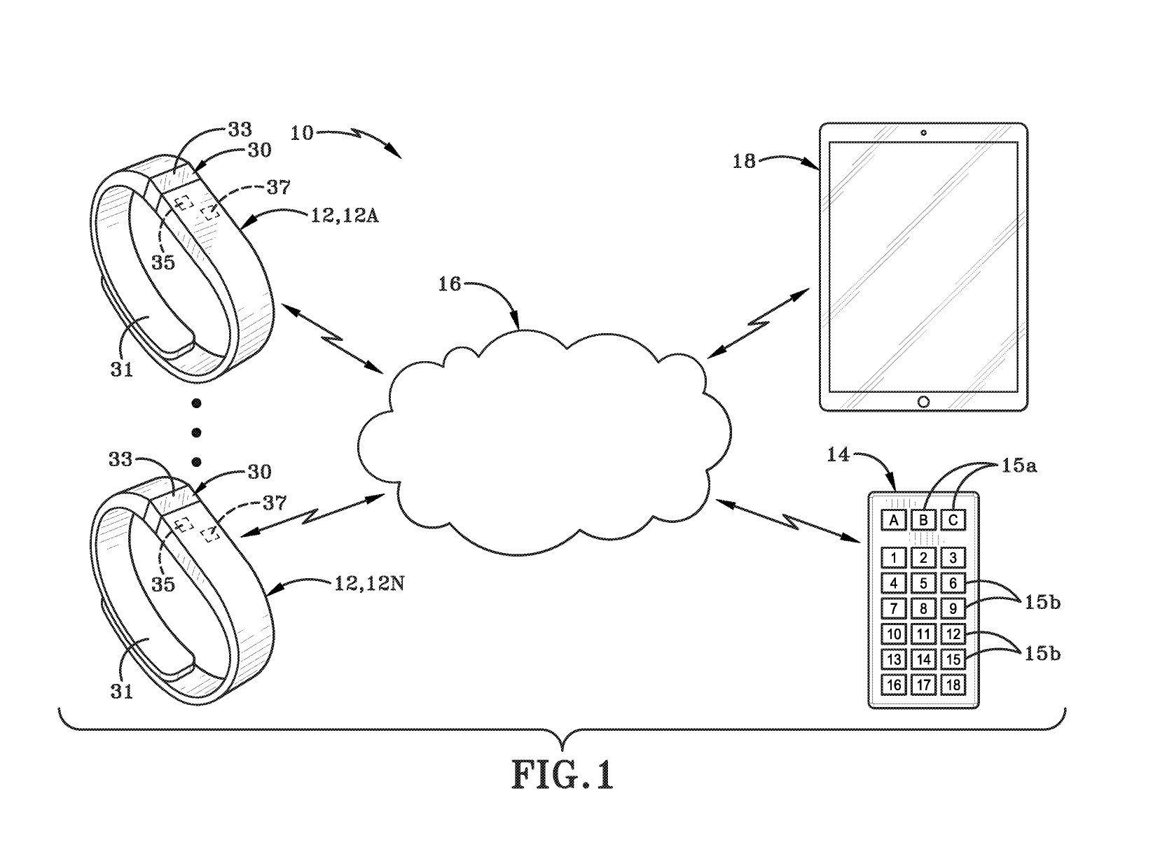

The present disclosure provides a system and method for student engagement in a classroom environment which is intended to be used in order to foster student learning in the classroom. The student engagement system of the present disclosure is indicated in FIG. 1 generally at 10. The student engagement system 10 may include a plurality of wearable electronic devices 12, a handheld teacher remote controller 14, a connecting network 16, and a computing device, such as a tablet, 18.

At the outset, it is noted that for the present disclosure student engagement is distinct from student behavior which is different than student motivation. Behavior is the “how” student acts; engagement is typically the “what” behind the act; and motivation is typically the “why” a student acts. “Student engagement” is predicated on the position that student learning improves when students are inquisitive, interested, or inspired, and that learning tends to suffer when students are bored, dispassionate, disaffected, behaving inappropriately or otherwise “disengaged.” Student engagement also refers to the degree of attention, curiosity, interest, optimism, and passion that students show when they are learning or being taught, which extends to the level of motivation they have to learn and progress in their education. This is usually best determined by the teacher in the classroom as there is typically a strong interpersonal relationship between the student and the teacher. In one sense, it may be possible for a student to be “engaged” while still not behaving in an appropriate manner. Student engagement also refers to the role that certain intellectual, emotional, physical, and social factors play in the learning process and social development. For example, there are connections between “non-cognitive factors” or “non-cognitive skills” (e.g., motivation, interest, curiosity, responsibility, determination, perseverance, attitude, work habits, self-regulation, social skills, etc.) and “cognitive” learning results (e.g., improved academic performance, test scores, information recall, skill acquisition, etc.).

As depicted in FIG. 1, a first wearable electronic device 12A is configured to be worn around the wrist of a first student. Within system 10 there may be a plurality of wearable electronic devices 12 which is generally as 12N, wherein N is equal to the total number of students in a classroom setting. Each wearable electronic device 12 includes opposing straps 31 configured to be repeatably connected and disconnected to enable the wearable electronic device 12 to be releasably secured around the wrist of a student. Further, each wearable electronic device 12 includes a display panel 30 which is configured to provide feedback information pertaining to student engagement when worn by the student.

Display panel 30 may be an electronic display including a plurality of lights configured to be illuminated. In one example, display panel 30 may include a large number of pixels so as to enable the display panel 30 to display text in the event textual displays are desired. In another example, and as will be described in greater detail below, display panel 30 may include at least three Light Emitting Diodes (LED or LEDs). Display 30 is carried by a housing 33 sized to be generally wrist-worn adapted to display a level of student engagement. Housing 33 is carried by straps 31 so as to effectuate the wrist wearing capabilities of device 12.

The wearable electronic device 12 includes internal communication logic powered by a rechargeable battery that is configured to communicate through network 16 with the computing device or computer 18 and the teacher's remote controller 14. Communication logic 35 establishes electrical network connectivity and may be selected from an exemplary group comprising a blue tooth connection, a wireless internet connection, a wired internet connection, and a 3G/4G connection. In another example, the wearable electronic device 12 may include at least one non-transitory computer readable storage medium 37 having instructions encoded thereon that when executed by one or more processors carried by the housing perform operations to display the level of student engagement in the display, the operations including: (i) receive a plurality of time frames including a first engagement period and a second engagement period from a distinct and remote controller 14 generated via teacher 13 input; (ii) receive signals generated via teacher 13 input from the remote controller 14, wherein the signals are based on student engagement observed by the teacher 13 during the first engagement period, and the signals represent positive feedback and no signals are sent representing inappropriate behavior; and (iii) notify the student 11 by altering the display 30 to represent a decline in student engagement in the event the teacher has not observed sufficient student engagement during the engagement period and has not generated the signal representing positive student engagement; and wherein student engagement is independent and distinct from student behavior, and student engagement includes a degree of attention, curiosity, interest, optimism, and passion shown by a student when learning or being taught.

Communication logic 35 may also include a receiver carried by the housing 33 to receive signals from the remote controller 14, wherein if the receiver of communication logic 35 receives a positive student engagement signal from the controller 14 during a first engagement period, then all three LEDs (described in greater detail below) remain illuminated in a first color, and if the receiver does not receive the positive student engagement signal from the controller 14 during the first engagement period, then one of the LEDs will illuminate in a second color until the receiver receives the positive student engagement signal generated via teacher input into the controller 14.

Wearable electronic device 12 may further include timing logic in operative communication with the at least one non-transitory computer readable storage medium 37 having instructions encoded thereon configured to initiate a countdown timer during which the student is to maintain his or her student engagement. The countdown timer may or may not be viewable by the student wearing device 12 in display 30. In some instances, the clock measures the engagement periods so as to determine whether a positive engagement signal has been received during an engagement period.

Wearable electronic device 12 may further include a plurality of one or more sensors within the wearable electronic device 12 which are configured to detect movement of the student while wearing the wearable electronic device 12. The sensors may collect data based on physical movements of the student. Additionally, the initiation of data collection when received from the sensors will initiate during a detectable motion intensity signature. An input to a trigger algorithm may come directly or indirectly from the sensor output. For example, the input may be direct output from an accelerometer or it may be processed accelerometer output. An exemplary trigger can be a student raising his or her arm in class when prompted by the teacher to answer a question. In such a condition, the motion intensity detected by the motion sensor within wearable electronic device 12 can initiate a data collection sequence from the sensor coupled with the communication logic transmits this information over the network to computing device 18 to show that the student raised his or her hand during class lecture.

However, motion signatures are not a required portion of the invention. As indicated previously, movement or physical behavior is not always indicative of student engagement. So while the wearable electronic device 12 is able to initiate data collection based on a movement signature, it is not required, in as much as the student engagement is cued from positive behaviors selectively input from the remote 14 operated by the teacher 13.

In some embodiments, information collected from the sensors of wearable electronic device 12 may include a record of information for a previous period, for example, one day, two days, or three days, or for a fixed time interval such as once per hour, once per half hour, etc. Wearable electronic device 12 may further include artificial intelligence capable of learning such that digital information may be used to detect behavior signatures from a specific student compared and averaged to the rest of the students in the classroom or to predict the likelihood of certain activity levels for a student relative to his or her classmates. For example, if a student is more apt to be inactive or have a lapse of student engagement at certain times of the day relative to other students, the teacher may be alerted that said student needs specific attention during those time periods.

In some embodiments, algorithms operating in the frequency domain are embodied in wearable electronic device 12 and are used to determine the number of times (i.e., the frequency) a student is engaged over a period of time. One problem that middle school and high school aged students tend to have is that they do not stay engaged for significant periods of time during the classroom experience. The frequency logic contained within wearable electronic device 12 can count the number of times a student is engaged per day or per hour or per half hour.

By way of non-limiting example, if a student has not been engaged for the past half hour period, display indicator 30 identifies such lack of engagement and provides feedback to the student so that they may correct his or her engagement. In some implementations, wearable electronic device 12 may include one or more vibramotors (also referred to herein as vibrators or simply as vibrating devices) for communicating information to the student. For example, a processing unit can utilize the vibramotors to communicate one or more alarms, achievement goals, progress indicators, or inactivity indicators to the student wearing the wearable electronic device 12.

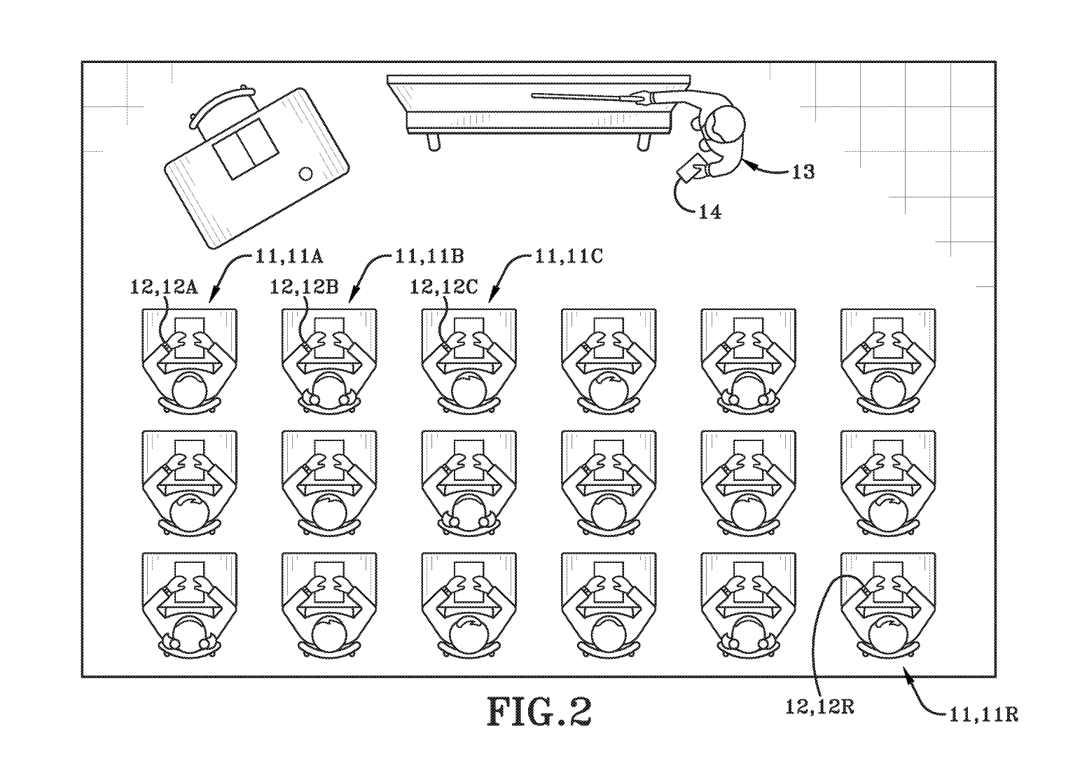

With continued reference to FIG. 1, the teacher's remote control 14 is a handheld electrically rechargeable powered device comprising one or more input actuators, which are shown in the form of keys 15a, 15b. The remote controller 14 is configured to be handheld by the teacher 13 (FIG. 2) and the teacher can input an entry through one of the keys 15a, 15b and that input entry may be connected through network 16 to either the computing device 18 or directly to one of the students' wearable electronic devices 12. Remote control 14 is preferably embodied as a mobile application executed on a smartphone or tablet computer. In this instance the keys 15a, 15b would be digital buttons configured to be pressed via a touchscreen on the smartphone. In this instance, the controller would carry inside it a non-transitory computer readable storage medium having instructions encoded thereon that when executed by one or more processors in the controller 14 would implement operations to send a signal from the controller through network 16 to one or all of the wearable electronic devices 12.

A first set of keys identified generally at 15a may correspond to a predetermined and selectively set communication notification by the teacher. For example, key 15a, labeled “A,” may be associated with a communicating activity that the teacher desires to notify the student. For example, key 15a, labeled “A,” may be associated with a student asking a relevant question to the topic that is being taught. Another key 15a, labeled “B,” may be associated with providing valuable comments on the lesson that is being discussed. Additionally, another key 15a, labeled “C,” may be associated with a student that is behaving properly or is otherwise on task. Alternatively, rather than keys 15a being labeled “A”, “B”, or “C”, keys 15a may include icons that correspond to behavior, such as a smiley-face, a green check mark, or a red X. First set of keys 15a may be selective programmed or selectively set by either the teacher 13 or the manufacturer of device 14. It is envision that teacher programming of keys 15a would be preferable inasmuch as the teacher knows the best types of student response indicative of engagement. When the teacher programs keys 15a, they will also show in the student portal on device/tablet 18.

The second set of keys 15b are associated with numerical indicators that correspond with a respective wearable electronic device 12 worn by a student. The teacher may select one of these second set of keys 15b in conjunction with one of the keys from the first set of keys 15a. Thus, for example, the teacher may press the first key (labeled “1”) of the second set of keys 15b, then the corresponding letter “A” from the first set of keys 15a. This input from the teacher would indicate that the first student wearing the wearable electronic device, such as wearable electronic device 12A, would be reinforced with positive engagement behavior for asking a relevant question to the lecture at hand.

In accordance with an aspect of the present disclosure, utilizing the first set of keys 15a with positive reinforcement behaviors has been to result in better learning capabilities for the students rather than a demerit system which is indicated in some of the prior art. While it is not referred to herein, however it is entirely possible that one of the first set keys 15a could be associated with an improper behavior such that the teacher could indicate to either the student wearing the wearable electronic device 12, or a third party such as a parent observing the student on computing device 18, that his or her behavior was not acceptable.

Furthermore, while not shown in FIG. 1, the remote controller 14 could clearly be enabled to operate with other keys and other input functions. For example, the remote controller 14 may be equipped with a “select all” key which would input every student in the second set of keys 15b with a certain behavioral item identified from the first set of keys 15a. For example, the teacher could input the select all key and then an action from this first set of keys 15a to indicate that every student in the group is performing on task.

A classroom setting is indicated generally in FIG. 2. Particularly, a plurality of students is identified generally as 11. A first student 11A wears the first wearable electronic device 12A around his or her wrist. A second student 11B wears the second wearable electronic device 12B around his or her wrist. This pattern continues to the eighteenth student 11R wearing the eighteenth wearable electronic device 12R around his or her wrist. The eighteen students depicted in FIG. 2 are not intended to be limiting and are rather an exemplary number of students in a classroom and the system 10 may be scaled to fit any number of students within a classroom setting.

It is envisioned that the classroom identified in FIG. 2 is either a middle school classroom or a high school classroom including students 11 who are able to regulate their own behaviors but need prompted or reminded to engage and participate in the lecture performed by the teacher 13. System 10 provides a solution for students 11 who often do not realize that they are not paying attention in class or participating in class which will help them gain a better understanding of the subject material that is being taught by teacher 13. System 10 enables a student to receive positive reinforcement from teacher 13, which is advantageous over the prior art because middle school and high school aged students are typically reluctant to respond to teacher prompts to participate or ask questions, especially when the student 11 is singled out. System 10 operating within the classroom environment of FIG. 2 prevents the teacher 13 from having to actively demand student participation and rather provides a confidential way for students to receive engagement feedback based on their behaviors which are input to the system by teacher 13 through remote controller 14. Additionally, the classroom may be an elementary school, such that students are in a range from kindergarten to about fifth grade.

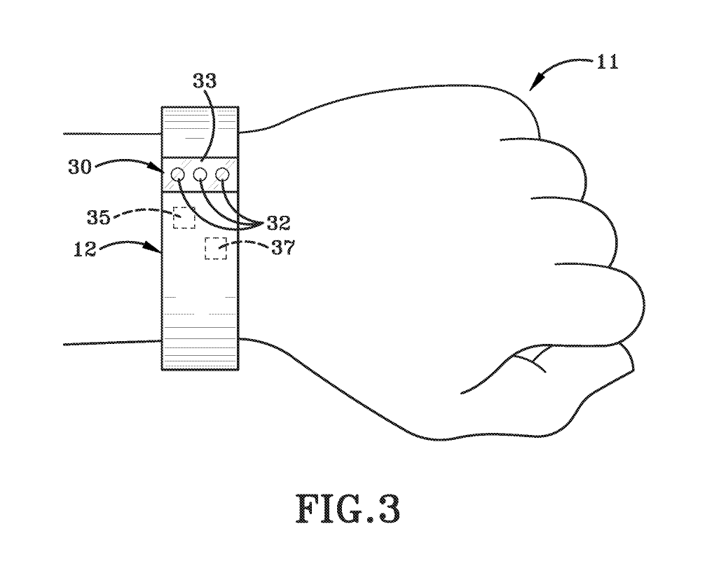

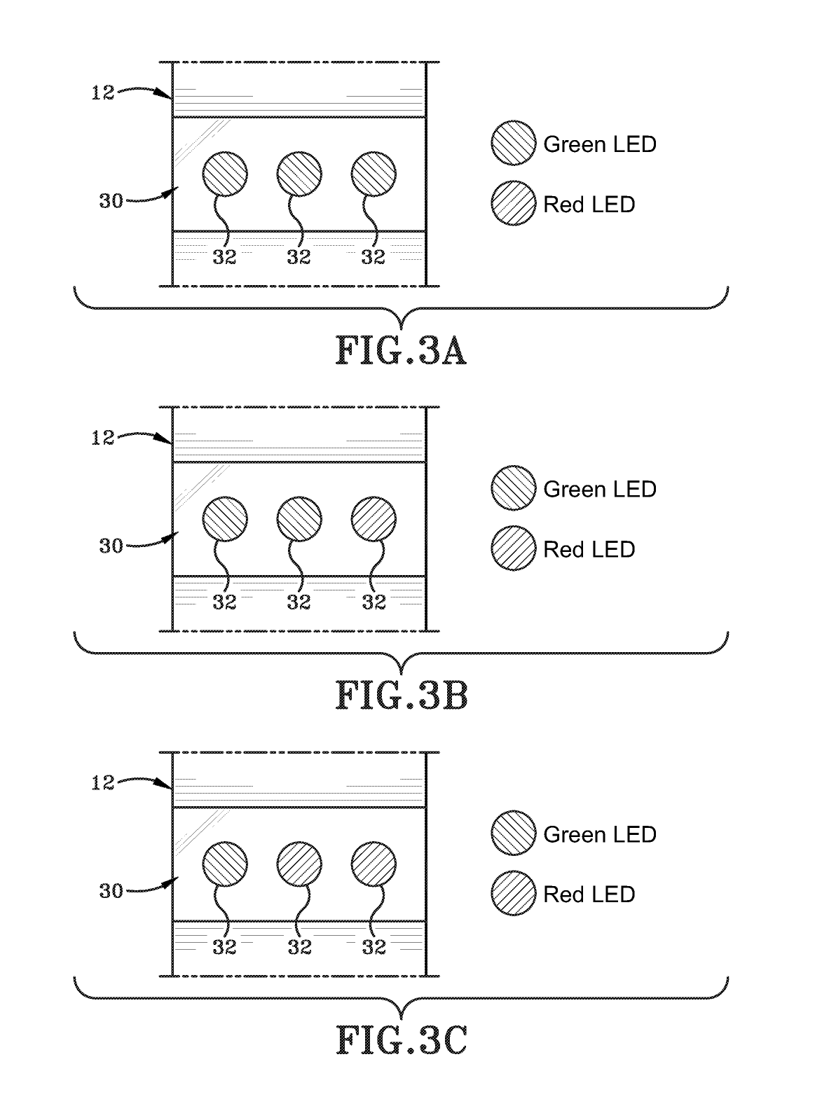

As depicted in FIG. 3, the graphical display 30 of wearable electronic device 12 may include one or more light emitting diode (LED) markers or identifiers 32. The LEDs 32 represent the student engagement for a selected time period. For example, remote controller 14 may enable the teacher 13 to select a desired engagement time period. Usually, engagement time periods are short within five to eight minutes. As time continues within a given engagement period, the LEDs 32 will change colors to indicate that the student 11 needs to contribute to the classroom discussion. For example, as indicated in FIG. 3A, when the student is fully engaged each of the LEDs 32 may be lit green. After one-third of the selected time period has expired without the student engaging within the classroom, one of the LEDs 32 may turn red as is indicated in FIG. 3B. In this instance, if the time period selectively set by the teacher 13 was six minutes, then the single red LED corresponds to a two-minute time period for which the student was not engaged. Stated otherwise, at least one light 32 in the display 30 illuminates in response to signals generated from the remote controller 14, wherein the light 32 remains illuminated during the engagement period and either darkens or changes color after the illumination period if no signal representing positive student engagement was received by the wearable device from the remote controller operated by the teacher.

With continued reference to FIG. 3C, when a single LED 32 is lit red, the student may have the opportunity to raise his or her hand, ask a question, or otherwise earn an input from the teacher 13 from the first set of keys 15a corresponding to a desirable student engagement as subjectively determined by the teacher.

If the student 11 participates and engages with quality responses or questions, then the teacher would indicate as much by pressing the keys on remote controller 14. When the teacher 13 indicates that the student 11 has achieved an engagement sufficient to the teacher's determination, the input through remote controller 14 from teacher 13 will be sent through network 16 to the corresponding wrist wearable electronic device 12A worn by student 11 to change the red LED 32 back to green.

The LEDs 32 changing between green and red are not intended to be limiting. Rather, the present disclosure contemplates that any LED colors are useful. However, middle school and high school aged students naturally associate green as positive and red as negative which is regularly reinforced as one having ordinary skill in the art would clearly understand and foresee.

As indicated in FIG. 3C, if a student has not been engaged for two-thirds of the selected time period, then two of the LEDs 32 will turn from green to red. At this instance, it is the intent of system 10 that the student will be encouraged to engage in classroom participation because of the impending three red LEDs 32 that students naturally associate with negative feedback or negative performance. The visual identification of the LEDs 32 changing color provides immediate and prompt feedback that many students will need to participate, and more importantly learn the lesson that is being taught by teacher 13. Furthermore, the vibramotors associated with wearable electronic device 12 may also be activated when the LEDs 32 all turn red.

With continued reference to FIG. 3A-3C, at least one light in display 30 is one of three LEDs 32. Further, an engagement period is one of three engagement periods, and wherein each one of the LEDs is associated with one engagement period.

In some instances, it may be advantageous for the teacher 13 to suspend or disable monitoring the student engagement once a lecture or activity warranting use of has ended. Students' wearable electronic devices 12 may be remote controlled and overridden by the teacher 13 to be placed into “stand by” mode. Once a class is done for the day or period, the teacher can “disable” students' devices 12. In one example, the display shows the time and date. Keys may be included in remote 14 to enable, disable, and place devices 12 into stand by mode.

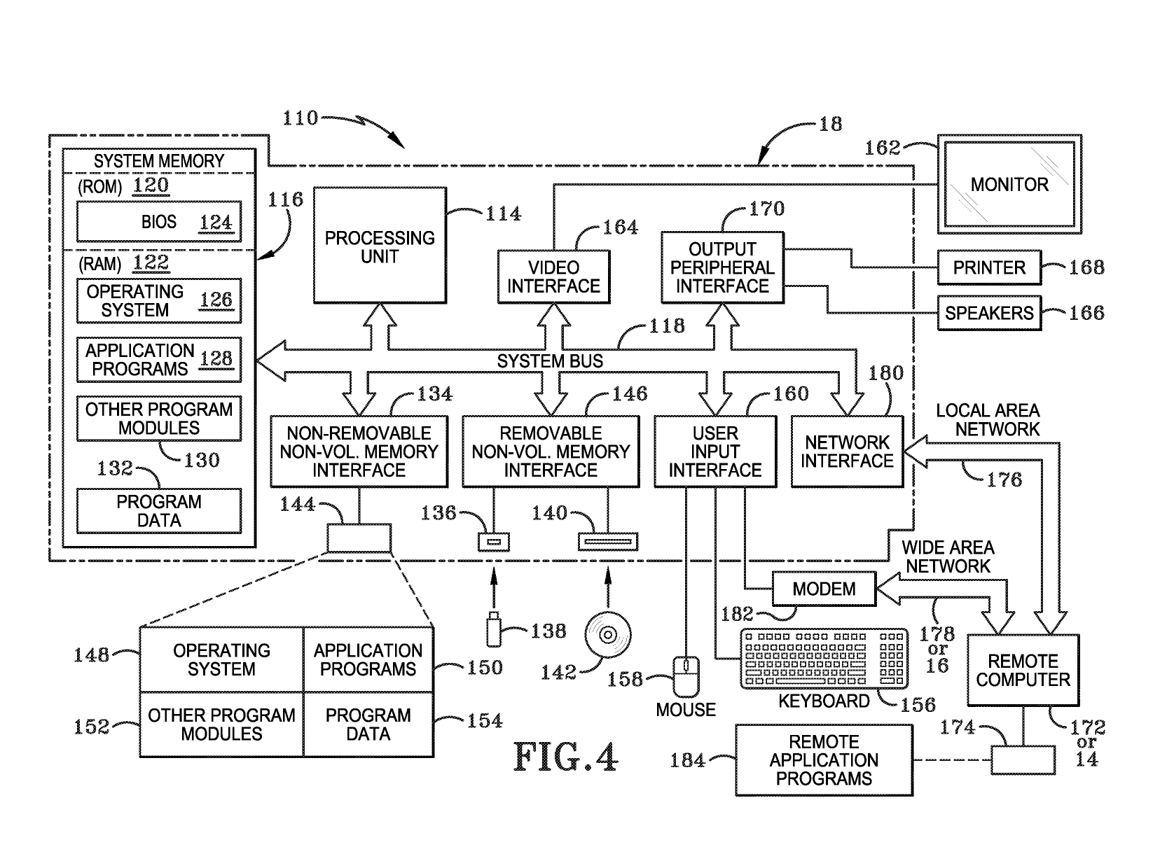

FIG. 4 depicts an exemplary computing system environment 110 which can define a portion of the system 10. The computing system environment 110 is provided to exemplify a manner in which the claimed method, programmed memory, and apparatus may be implemented. The computing system environment 110 is only one example of a suitable computing environment and is not intended to suggest any limitation as to the scope of use or functionality of the invention. Neither should the computing environment 110 be interpreted as having any dependency or requirement relating to any one or combination of components illustrated in the exemplary operating environment 10.

The claimed methods, programmed memory and apparatus are operational with numerous other general purpose or spatial purpose computing system environments or configurations. Examples of well-known computing systems, environments, and/or configurations that may be suitable for use with the invention include, but are not limited to, personal computers, server computers, hand-held or laptop devices, smartphones (such as an Apple iPhone or a Samsung Galaxy or the like), multiprocessor systems, microprocessor-based systems, set top boxes, programmable consumer electronics, network PCs, minicomputers, mainframe computers, distributed computing environments that include any of the above systems or devices, and the like.

The claimed methods, apparatus and programmed memory may be described in the general context of computer-executable instructions, such as program modules, being executed by a computer. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

With reference to FIG. 4, an exemplary system for implementing the claimed methods, apparatus and programmed memory includes a general purpose computing device in the form of a computer 18 (which may also be referred to as tablet 18). Components of computer 18 may include, but are not limited to, a processing unit 114, a system memory 116, and a system bus 118 that couples various system components including the system memory to the processing unit 114. The system bus 118 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 18 typically includes a variety of computer readable media. Computer readable media can be any available media that can be accessed by computer 12 and includes both volatile and nonvolatile media, removable and non-removable media, and at least one non-transitory computer readable storage medium (plural media). By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. This may also include non-transitory computer readable storage media. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 18. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer readable media.

The system memory 116 includes computer storage media in the form of volatile and/or nonvolatile memory such as read only memory (ROM) 120 and random access memory (RAM) 122. A basic input/output system 124 (BIOS), including the basic routines that help to transfer information between elements within computer 18, such as during start-up, is typically stored in ROM 120. RAM 122 typically includes data and/or program modules that are immediately accessible to and/or presently being operated on by processing unit 114. By way of example, and not limitation, FIG. 1 illustrates operating system 126, application programs 128, other program modules 130, and program data 132.

The computer 18 may also include other removable/non-removable, volatile/nonvolatile, transitory/non-transitory computer storage media. By way of example only, FIG. 4 illustrates a hard disk drive 134 that reads from or writes to non-removable, nonvolatile magnetic media, a drive 136 that reads from or writes to a removable, nonvolatile magnetic disk or USB flash drive 138, and an optical disk drive 140 that reads from or writes to a removable, nonvolatile optical disk 142 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 144 is typically connected to the system bus 118 through a non-removable memory interface such as interface 134, and drive 136 and optical disk drive 140 are typically connected to the system bus 118 by a memory interface, such as interface 146.

The drives and their associated computer storage media discussed above and illustrated in FIG. 4, provide storage of computer readable instructions, data structures, program modules and other data for the computer 18. In FIG. 4, for example, hard disk drive 144 is illustrated as storing operating system 148, application programs 150, other program modules 152, and program data 154. Note that these components can either be the same as or different from operating system 126, application programs 128, other program modules 130, and program data 132. Operating system 148, application programs 150, other program modules 152, and program data 154 are given different numbers here to illustrate that, at a minimum, they are different copies.

A user may enter commands and information into the computer 18 through input devices such as a keyboard 156 (which may also be a touchscreen keypad or keyboard) and pointing device 158, commonly referred to as a mouse, trackball or touch pad. Other input devices (not shown) may include a touchscreen, buttons, individual keys, microphone, joystick, game pad, satellite dish, scanner, or the like. These and other input devices are often connected to the processing unit 114 through a user input interface 160 that is coupled to the system bus 118, but may be connected by other interface and bus structures, such as a parallel port, game port or a universal serial bus (USB). A touchscreen monitor 162 or other type of display device is also connected to the system bus 118 via an interface, such as a video interface 164. In addition to the monitor 162, computers may also include other peripheral output devices such as speakers 166 and printer 168, which may be connected through an output peripheral interface 170.

The computer 18 may operate in a networked environment using logical connections to one or more remote computers, such as a remote computer 172 (which may also be the remote 14 when the remote 14 is implemented as a smartphone). The remote computer 172 may be a personal computer, a server, a router, a network PC, a peer device, a smartphone such as controller 14 or other common network node, and typically includes many or all of the elements described above relative to the computer 18, although only a memory storage device 174 has been illustrated in FIG. 4. The logical connections depicted in FIG. 1 include a local area network (LAN) 176 and a wide area network (WAN) 178 (which is generally shown in FIG. 1 as network 16), but may also include other networks. Such networking environments are commonplace in offices, enterprise-wide computer networks, intranets and the Internet.

When used in a LAN networking environment, the computer 18 is connected to the LAN 176 through a network interface or adapter 80. When used in a WAN networking environment, the computer 18 typically includes a modem 182 or other means for establishing communications over the WAN 178 (or generally network 16 of FIG. 1), such as the Internet. The modem 182, which may be internal or external, may be connected to the system bus 118 via the user input interface 160, or other appropriate mechanism. In a networked environment, program modules depicted relative to the computer 18, or portions thereof, may be stored in the remote memory storage device. By way of example, and not limitation, FIG. 4 illustrates remote application programs 184 as residing on memory device 174. It will be appreciated that the network connections shown are exemplary and other means of establishing a communications link between the computers may be used.

Inasmuch as computing environment 110 has been described, it should be well understood that various components of the present disclosure such as the controller 14, the wearable electronic device 12 and the central server or data base may interconnect with the computer or tablet 18 of this type. Furthermore, when controller 14 is implemented as a smartphone, it would include similar computing components to those identified above in FIG. 4 and is not repeated herein for brevity, however the structural and electrical similarities are to be understood.

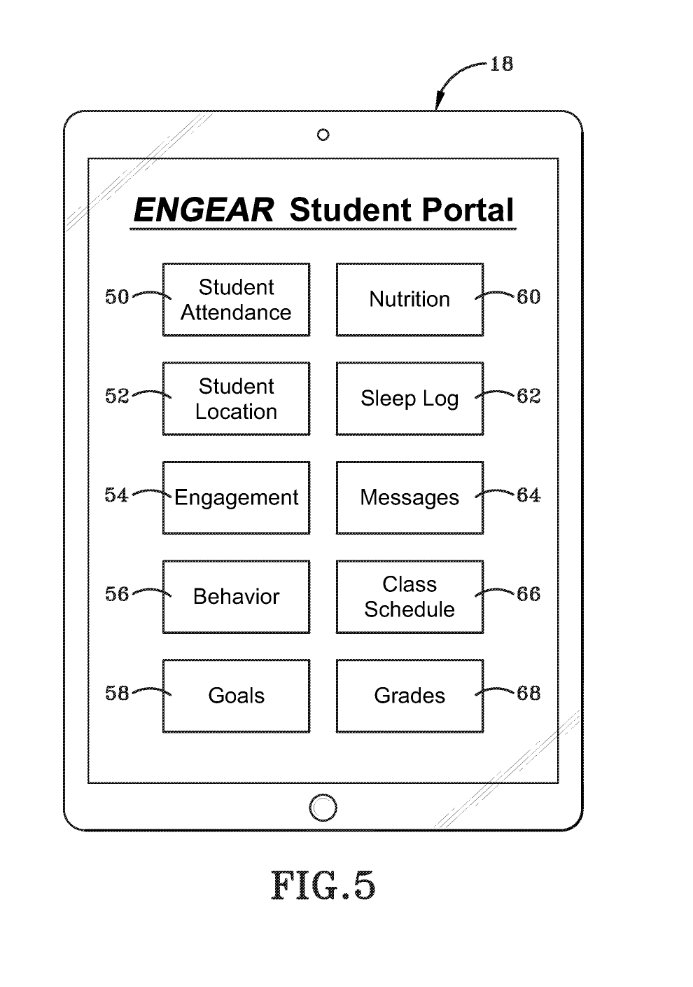

As depicted in FIG. 5, the computing device 18 is shown in the form of a tablet, however may take the form of a smartphone or a laptop computer. The computing device 18 shows a student portal on the monitor which is associated with a single wearable electronic device 12 that would have associated login credentials for private access of the student. The student portal may: track student attendance, which is shown generally at 50; provide access to the student location when the wearable electronic device 12 is equipped with GPS technology, which is shown generally at 52; provide a summary of the student's engagement during the lesson taught by the teacher, which is shown generally at 54; track the behavior of the student as input by the teacher, which is shown generally at 56; provide and track a list of selective or predetermined goals for student achievement, which is shown generally at 58; track and provide a nutrition section which may enable the student to track school meals and allow the student's parents to ensure that the student 11 wearing the associated wearable electronic device 12 has a proper meal for the day, which is shown generally at 60; provide a sleep log, which is common for wearable electronic devices 12, which is shown generally at 62; establish a message portal for the parents or student to communicate with teacher 13, which is shown generally at 64; provide an identification of class schedule which is shown generally at 66; and provide a portal for grades of the student in the teacher's classroom, which is shown generally at 68.

The student portal may be controlled by an administrator. The administrator may selectively set the level of access permitted to the student portal. For example, all teachers may be able to see every student portal. Alternatively, only a single teacher may be able to view only a portion of one student's portal.

The attendance system 50 for the student portal operates via communication logic between wearable electronic device 12 and either the remote 14 or a third-party networked computer managed by the school. The student's attendance may be monitored when they enter the school or a particular classroom. The attendance may automatically update based on the student's location within the building.

In one exemplary embodiment, a student 11 enters the building with wearable electronic device 12 and goes to his or her particular homeroom. The teacher 13 may then request the class to sync their wearable electronic devices 12 with a host computer, or remote 14 which may serve as the host computer when the remote is embodied as a smartphone or tablet computer. The teacher 13 should also take physical attendance to ensure the student matches the wearable electronic device recorded by computer 18.

The student attendance is then reported throughout the day based on the student's location. This may be accomplished by the GPS technology built into the wearable electronic device 12 and a Bluetooth connection across network 16 to one or more school host computers. This enables both the school and parents (through portal on remote device 18) to locate a student for any purpose including in an emergency situation.

Further, the student wearable electronic devices 12 may resync upon entry of a new classroom setting for older students who switch periods. Teachers will enter a different key code to access numbers corresponding to the students. To begin engagement monitoring, the teacher will enable as before and disable at the conclusion.

A parent, legal guardian, or other authorized third party can log onto the student's portal via device 18 and view daily attendance in attendance portion 50 or receive a notification in message portion 64 if the student is not at school for a full or half day. A record is kept as well as a more detailed record of the student's location during the day on an administrator site which may or may not be viewable in tablet computer 18, or the administrator site may be a remote access application linked to system 10 via network 16.

In one particular embodiment, if a student's wearable electronic device 12 is not present in a room (after change periods), an alert is sent to the teacher's device (an iPad, iPod, computer, or other device with software downloaded on). The teacher can then contact the office to alarm the school resource officer, the teachers, the parents, or another interested party. If an error has occurred, the teacher may affirm and continue with class.

The student engagement portion 54 of the student portal displays the teacher-monitored student engagement throughout the class period. This feature may be enabled once the teacher logs into his or her device 14 with a key code. The displayed student engagement follows the previously outlined steps with students' device having the interval bar across the display. Accordingly, in accordance with one aspect of the present disclosure, the display 30 on device 12 is representative of an identical representation in the student engagement portion 54 of the student portal (i.e., the portal has corresponding green and red lights). However, it is possible for the portal to have an alternate manner of displaying the same feedback information. For example, the student engagement portion 54 of the student portal may incorporate various graphs and tables or charts communicating the level of student engagement to the student. Additionally, student engagement portion 54 further may provide recommendations based on programmed algorithms. For example, student engagement portion 54 may include an algorithm to calculate the frequency of the non-engaged periods during a classroom activity. Based on the frequency of non-engaged periods, the student engagement portion 54 can generate encouraging remarks or indicators which increase in encouragement as the frequency of non-engaged periods increase. For example, if a student has only one non-engaged period during the class room activity/lecture, the algorithms of student engagement portion 54 may recognize that this was only a minimal lapse of student engagement and generate a notification or message to indicate that the student only is performing very well and that there was only a small lapse of student engagement. In another example, if the same student has multiple periods of non-engagement, then the student engagement portion 54 may recognize this as a more serious representation of non-engagement and generate a notification or message to indicate that the student appears to be or may be at risk of falling behind because of their number of non-engaged periods. Student engagement portion 54 may also compare the number of engaged period and non-engaged periods and display the same relative to an average number of engaged periods and non-engaged period of the entire class to thereby enable the student to see his or her relative student engagement levels relative to his or her peers/classmates. One embodiment may provide that some students may be encouraged to increase engagement levels if they can see how they perform relative to classmates.

The message system 64 for the student portal operated via message logic on computing device 18 may enable an authorized user, such as a parent, to send a signal carrying an electronic message from the computing device to the graphic display 30 on one of the wearable electronic devices 12. The wearable electronic device 12 may be coded with logic that would enable the signal to flash across the display in real time to enable the parent from a remote location to encourage the student to have a higher level of engagement. In this instance, the display 30 would be incorporated with an LCD screen to allow the identification of the text message scrolling thereacross. It is contemplated that a parent would send either a positive message to encourage good student engagement or an encouraging message to correct insufficient student engagement. For example, a parent may send from the device 18 through the message portal a message such as “great work today, keep up the good work,” or an encouraging statement like “don't let one wrong answer get you down.”

The nutrition log 60 within the student portal of computing device 18 may be utilized in conjunction with a student's online account at the school. The food that they purchase during a lunch could automatically be synced with the student portal operated on device 18 and reported to his or her account for at least one parent or legal guardian to view. The nutrition log could contain both a summary of a lunch money account balance of how much money the student has spent on recent school lunches as well as the nutritional information about the food that the student has consumed.

Additionally, information from the cafeteria can be synced with the wearable electronic device and the student portal on computing device 18. When a student passes through the lunch line, his or her purchases may be sent to the device and account (portal). This will provide parents with the knowledge of what their child is buying and consuming at school, as well as a debit/credit account balance information. Furthermore, device 12 may be utilized to pay for school lunches. In this instance, the device 12 may be equipped with an RFID transmitter and the school can be equipped with an RFID receiver. When the student walks past the RFID receiver with their food, the transmitter may generate a signal (which is received by the receiver) to debit the student's lunch money account.

The student location 52 portion of the student portal for computing device 18 may rely on either GPS sensors within the device or other tracking means associated with identifying the presence of an item, such as an RFID chip or transmitter (the same one or a different one from the RFIRD transmitter used to purchase lunch, as explained above) which may be incorporated into an antenna mounted in the school. Together, these mechanisms could increase school safety to account for the students and assist the teachers with the necessary roll call, especially during an emergency situation. Otherwise, in non-emergency situations, this may provide an automated system for the teacher to establish which students are present and verify attendance. Furthermore, the student location may be provided to the portal operated on computing device 18 for the parents to verify that the students are in fact at school and not at another location.

The sleep log 62 portion of the student portal operating on the computing device 18 may provide data on the student's sleep cycle to indicate how much sleep has been acquired the night before or over a selected time period, such as the past week. This enables parents to increase accountability for student engagement that has a tremendous influence on academic performance. Studies indicate that sleep is directly tied to student engagement. Thus, it is critical for middle school and high school aged students to receive the proper amount of sleep.

In operation, and in accordance with one exemplary aspect of the present disclosure, system 10 fosters student engagement system to encourage learning in the classroom. System 10 utilizes a wearable electronic device 12 to encourage student ownership of learning by visually (or tactically) revealing to students how active or engaged they are at a specific time or over a time period (i.e. class or day). Each device 12 corresponds to a student number (based on number in classroom and connected to student id). The teacher 13 has a remote 14 (i.e., a mobile application executed on a smartphone) size device with a key board featuring numbers (i.e., keys 15b) and several alphabet letters on the top (i.e., keys 15a). Each letter on keys 15a denotes a category the teacher might be specifically tracking (ex. questioning, commenting, or on-task performance). Teachers can choose to utilize all or several categories or only focus on tracking one trait or characteristic of student engagement. Upon demonstrating a specific positive student engagement, the teacher is able to quickly enter the student number associated with one of the keys 15b and activity letter from key 15a while continuing the lesson. A “select all” key may also located on the remote to allow for more efficient entry in instances where all students are working on a group activity or individual assignment. In one operational embodiment, remote 14 is a mobile application downloaded on a smartphone or table computer, if preferred over a remote.

In operation, the wearable electronic device 12 then displays a green bar formed by a series of illuminated green lights representing ongoing student engagement for a time period (5-8 minutes or longer if desired by teacher). As the time continues, the display bar will change colors and eventually turn to red indicting the student needs to contribute to the class again. The engagement bar is divided into three sections with the total time period properly divided dependent on length chosen by the teacher or automatically used. In the same manner, one example provides three engagement periods, namely, a first engagement period, a second engagement period, and a third engagement period. For instance, if a teacher selects to employ eight minute periods each section on the green bar would represent 2.66 minutes. Therefore, after five minutes of not questioning or working, a student's bar would only show one section of green. This visually provides immediate prompting and feedback that many students need to participate and more importantly to learn. Other embodiments may provide that each engagement period is about 10 minutes each (30 minutes total). Other embodiments may provide that each engagement period is about 8 minutes each (24 minutes total). Other embodiments may provide that each engagement period is about 6 minutes each (18 minutes total). Other embodiments may provide that each engagement period is about 5 minutes each (15 minutes total). Other embodiments may provide that each engagement period is about 4 minutes each (12 minutes total). Other embodiments may provide that each engagement period is about 9 minutes each (3 minutes total).

In one non-limiting example, system 10 may be used with middle school and high school students (about 11-18 years old) who are able to regulate their bodies/actions but need prompted or reminded to do so in class. However, in another example, system 10 is used with elementary students (grades K-5; approximately ages 5-10). An exemplary advantage of using system 10 with elementary or primary school-aged children is to provide another tool for the school to teach the students how to regulate their engagement since young children already recognize that green lights on the device 12 correspond to positive actions and red lights on device 12 correspond to less favorable actions (or even negative actions). Another advantage of using system 10 with elementary school aged children is a safety factor for providing their location in addition to engagement understanding.

Many students often do not realize that they are not paying attention in class or even participating to help them gain a better understanding. This said student(s) is at a disadvantage when it comes to achievement. Older students are also often reluctant to respond to teacher prompts to participate or ask questions, especially when they are singled out. System 10 prevents teachers from having to push for student participation.

Furthermore, anxious parents often say that their child tells them they ask questions and “try” in class but just do not “get it.” Yet, in reality the student does not participate in lessons or ask questions. System 10 provides an easy manner to track student engagement, which may be independent from student behavior, and have valuable conversation between the teacher, student, and parents. Much like the fitness bands, such as FIT BIT®, the wearable electronic device 12 corresponds to an account online, which is accessible through computing device 18 display having an individual portal associated with login information for each student. The account would provide a place for the data to be viewed and discussed by both the student and the teacher, as well as parents and other administrators or service providers. The communicative logic 35 establishes the network connectivity effectuating the link between the device 12 and the portal on computer 18.

The present disclosure relates to a student engagement system for identifying and displaying information to students in a classroom in real time (without perceivable delay as perceived by the student) for increasing student engagement. It may be possible for the method of increasing student engagement to be implemented in other ways.

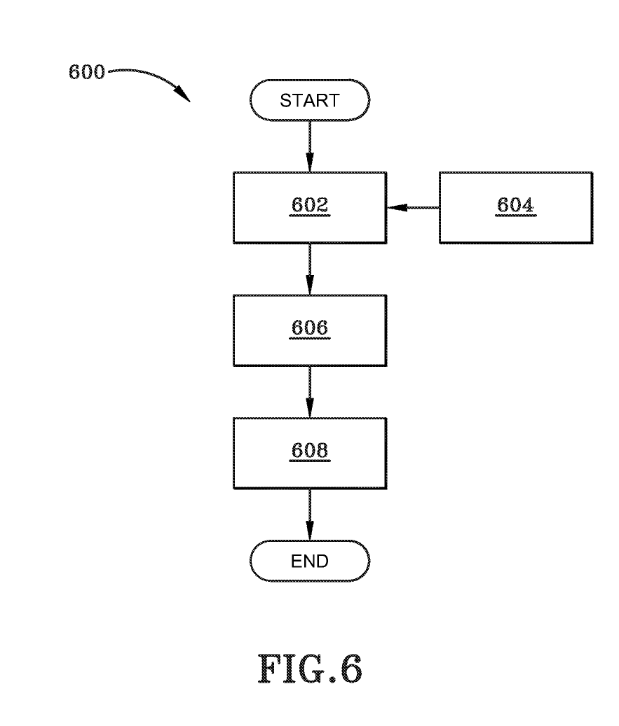

FIG. 6 depicts an exemplary method, shown generally at 600, in accordance with one aspect of the present disclosure. Method 600 is a method for student engagement. The method 600 includes receiving a wireless signal carrying instructions for execution by processors housed in the device 12 worn by a student 11 in a classroom, shown generally at 602. The method 600 may also include wherein the signal is generated, via teacher input, in the remote controller 14 based on student engagement during a classroom activity and student engagement is determined subjectively by a teacher, shown generally at 604. The method 600 includes executing the instructions via processors in the device 12 worn by the student 11, wherein the instructions include operations to notify the student 11 wearing the device 12 of his or her student engagement in learning irrespective of student behavior, shown generally at 606. The method 600 includes effecting an increase of student engagement based on the operations that notified the student of his or her engagement in learning irrespective of student behavior, shown general at 608.

Method 600 may further include receiving a plurality of time frames from the remote controller 14 operated by the teacher 13, wherein the plurality of time frames includes a first engagement period and a second engagement period. Method 600 may further include determining whether a first signal of positive student engagement has been received by device 12 during the first engagement period; and if the first signal has been received, then maintaining illumination of a light 32 during the second engagement period, and if no first signal is received then ceasing illumination of the light 32 during the second engagement period until the first signal is received, and the re-illuminating the light 32. Method 600 may further include processing the instructions in the device 12 worn by the student 11; activating at least one light 32 based on the instructions, wherein the light represents a level of student engagement subjectively determined through observation by the teacher 13; adjusting the illumination of the light in a display of the device after receiving a second signal indicating student engagement has returned after a period of non-engagement; and interrupting a period in which the student registers as non-engaged with a first engagement signal generated by the remote controller 14 and identifying the student 11 as engaged so as to illuminate the display 30 carried by the device 12 in a manner indicating engagement within the same period.

Method 600 may further include aggregating the levels of student engagement during a classroom activity and sending the levels of student engagement to a database accessible at a later time via a computer application, which may be executed on computer 18 and as described with respect to FIG. 5. Method 600 may further include collating and comparing levels of student engagement over multiple time periods, and providing a system recommendation of student engagement based on the collated and compared levels of student engagement. The system recommendation may be established through a comparative algorithm comparing the student engagement versus other students' engagement or the same student's engagement from a different time.

Method 600 may further include grouping periods of engaged activity to form engagement blocks viewable in the computer application, and grouping periods of non-engaged activity to form non-engagement blocks viewable in the computer application. Method 600 may further include recommending a category of “sufficiently engaged” or “insufficiently engaged” during the classroom activity based on the comparison of engaged grouping periods to non-engaged grouping periods. In one example, if the engaged grouping periods exceed the non-engaged grouping periods, then the student may be categorized as “sufficiently engaged” when viewed by a third party, such as a parent, in the computer application or portal. In one example, if the engaged grouping periods exceed the non-engaged grouping periods by a factor of at least 1.5× (i.e. engaged grouping periods is 1.5 times greater than non-engaged grouping periods), then the student may be categorized as “sufficiently engaged” when viewed by a third party, such as a parent, in the computer application or portal. In another example, if the engaged grouping periods exceed the non-engaged grouping periods by a factor of at least 2× (i.e. engaged grouping periods is 2 times greater than non-engaged grouping periods), then the student may be categorized as “sufficiently engaged” when viewed by a third party, such as a parent, in the computer application or portal. In another example, if the engaged grouping periods exceed the non-engaged grouping periods by a factor of at least 3× (i.e. engaged grouping periods is 3 times greater than non-engaged grouping periods), then the student may be categorized as “sufficiently engaged” when viewed by a third party, such as a parent, in the computer application or portal. In another example, if the engaged grouping periods exceed the non-engaged grouping periods by a factor of at least 5× (i.e. engaged grouping periods is 5 times greater than non-engaged grouping periods), then the student may be categorized as “sufficiently engaged” when viewed by a third party, such as a parent, in the computer application or portal. Additionally, the method may assist in diagnosing or confirming a medical diagnosis based on the number non-engagement periods. For example, the system 10 may assist in confirming a diagnosis of ADD or ADHD in student 11.

Method 600 may further include relaying the first engagement signal regarding the student engagement from the wearable device 12 to the database so as to enable the viewing of the teacher-generated signals via the computer application or portal. Method 600 may further include discretely notifying the student 11 of non-engagement so as to not cause distractions or draw the attention of other students to the non-engaged student.

Method 600 may further include actively observing for signals generated by the remote controller 14 by device 12. Alternatively, Method 600 may include passively observing for signals generated by the remote controller 14 by device. Further, Method 600 may provide wherein student engagement is independent and distinct from student behavior, and student engagement consists only of a degree of attention, curiosity, interest, optimism, and passion shown by the student when learning or being taught.

In yet another aspect, an exemplary embodiment of the present disclosure may provide a method of use from an exemplary school board's perspective comprising: providing a plurality of wearable student engagement monitors, such as device 12 including the display 30; providing a first wearable student engagement monitor to a first student, such as student 11A; providing a second wearable student engagement monitor to a second student, such as student 11B; providing a controller, such as controller 14, to a teacher, wherein the controller is in wireless communication with the plurality of wearable student engagement monitors; establishing, via teacher input into the controller, a first engagement period and a second engagement period; illuminating the display 30 during the first engagement period; requiring the teacher to indicate whether positive student engagement was present for each student during the first engagement period; wherein if the teacher indicates that student engagement was sufficient during the first engagement period, then the display remains fully illuminated during the second engagement period; wherein if the teacher indicates that student engagement was insufficient during the first engagement period, then the display is partially illuminated during the second engagement period or illuminated in a different manner so as to indicate a lapse in student engagement to the student; and wherein student engagement is independent and distinct from student behavior, and student engagement includes a degree of attention, curiosity, interest, optimism, and passion that students show when learning or being taught.

In yet another aspect, an exemplary embodiment of the present disclosure may provide a method of use from an exemplary teacher's perspective comprising: equipping students in a classroom with a wearable device, such as device 12 including display 30 and wireless communication logic 35; generating a first engagement time period and a second engagement time period and communicating the periods to the device 12; teaching a classroom activity; engaging with the students and observing student engagement irrespective of behavior; generating a signal, via teacher input, in a remote controller; sending the signal to each wearable device, wherein if the student was engaged during the first time period, then the signal instructs the device to illuminate a first amount of lights during the second time period, and if the student was not engaged during the first time period, based on the teacher's observation, then the signal instructs the device to illuminate less than the first amount of lights during the second time period or the same amount of lights in a different color so as to indicate non-engagement to the student. Again, in this scenario, student engagement may be independent and distinct from student behavior, and student engagement includes a degree of attention, curiosity, interest, optimism, and passion that students show when learning or being taught.

All definitions, as defined and used herein, should be understood to control over dictionary definitions, definitions in documents incorporated by reference, and/or ordinary meanings of the defined terms.

“Logic”, as used herein, includes but is not limited to hardware, firmware, software and/or combinations of each to perform a function(s) or an action(s), and/or to cause a function or action from another logic, method, and/or system. For example, based on a desired application or needs, logic may include a software controlled microprocessor, discrete logic like a processor (e.g., microprocessor), an application specific integrated circuit (ASIC), a programmed logic device, a memory device containing instructions, an electric device having a memory, or the like. Logic may include one or more gates, combinations of gates, or other circuit components. Logic may also be fully embodied as software. Where multiple logics are described, it may be possible to incorporate the multiple logics into one physical logic. Similarly, where a single logic is described, it may be possible to distribute that single logic between multiple physical logics.

Also, a computer or smartphone may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers or smartphones may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The various methods or processes (e.g., of designing and making the coupling structures and diffractive optical elements disclosed above) outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, USB flash drives, SD cards, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the disclosure discussed above. The computer readable medium or media can be transportable, such that the program or programs stored thereon can be loaded onto one or more different computers or other processors to implement various aspects of the present disclosure as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey a relationship between the fields. However, any suitable mechanism may be used to establish a relationship between information in fields of a data structure, including through the use of pointers, tags or other mechanisms that establish relationship between data elements.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. For example, embodiments of technology disclosed herein may be implemented using hardware, software, or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smartphone or any other suitable portable or fixed electronic device.

Also, a computer, such as computer 112, may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” or “algorithm” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.” The phrase “and/or,” as used herein in the specification and in the claims (if at all), should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures.

An embodiment is an implementation or example of the present disclosure. Reference in the specification to “an embodiment,” “one embodiment,” “some embodiments,” “one particular embodiment,” or “other embodiments,” or the like, means that a particular feature, structure, or characteristic described in connection with the embodiments is included in at least some embodiments, but not necessarily all embodiments, of the invention. The various appearances “an embodiment,” “one embodiment,” “some embodiments,” “one particular embodiment,” or “other embodiments,” or the like, are not necessarily all referring to the same embodiments.

If this specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the preferred embodiment of the disclosure are an example and the disclosure is not limited to the exact details shown or described.

What is claimed:

1. A student engagement method comprising:
receiving a wireless signal carrying instructions for execution by processors housed in a device worn by a student in a classroom, wherein the signal is generated, via teacher input, in a remote controller based on student engagement during a classroom activity and student engagement is determined subjectively by a teacher;
executing the instructions via processors in the device worn by the student, wherein the instructions include operations to notify the student wearing the device of his or her student engagement in learning irrespective of student behavior; and
effecting an increase of student engagement based on the operations that notified the student of his or her engagement in learning irrespective of student behavior.

2. The method of claim 1, further comprising:
receiving a plurality of time frames from the remote controller operated by the teacher, wherein the plurality of time frames includes a first engagement period and a second engagement period.

3. The method of claim 2, further comprising:
determining whether a first signal of positive student engagement has been received during the first engagement period;
if the first signal has been received, then maintaining illumination of a light during the second engagement period, and if no first signal is received, then ceasing illumination of the light during the second engagement period until the first signal is received, and then re-illuminating the light.

4. The method of claim 1, further comprising:
processing the instructions in the device worn by the student;
activating at least one light based on the instructions, wherein the light represents a level of student engagement subjectively determined through observation by the teacher;
adjusting the illumination of the light in a display of the device after receiving a second signal indicating student engagement has returned after a period of non-engagement; and
interrupting a period in which the student registers as non-engaged with a first engagement signal generated by the remote controller and identifying the student as engaged so as to illuminate a display carried by the device in a manner indicating engagement within the same period.

5. The method of claim 4, further comprising:
aggregating the levels of student engagement during a classroom activity and sending the levels of student engagement to a database accessible at a later time via a computer application;
collating and comparing levels of student engagement over multiple time periods, and providing a system recommendation of student engagement based on the collated and compared levels of student engagement;
grouping periods of engaged activity to form engagement blocks viewable in the computer application, and grouping periods of non-engaged activity to form non-engagement blocks viewable in the computer application.

6. The method of claim 5, further comprising
relaying the first engagement signal regarding student engagement from the wearable device to the database so as to enable the viewing of the teacher-generated signals via the computer application.

7. The method of claim 6, further comprising:
discretely notifying the student of non-engagement so as to not cause distractions or draw attention of other students to the non-engaged student.

8. The method of claim 1, further comprising:
actively observing for signals generated by the remote controller.

9. The method of claim 1, further comprising:
passively observing for signals generated by the remote controller.

10. The method of claim 1, wherein student engagement is independent and distinct from student behavior, and student engagement consists of a degree of attention, curiosity, interest, optimism, and passion shown by the student when learning or being taught.

11. A wearable device comprising:
a display carried by a housing sized to be generally wrist-worn adapted to display a level of student engagement;
at least one non-transitory computer readable storage medium having instructions encoded thereon that when executed by one or more processors carried by the housing perform operations to display the level of student engagement in the display, the operations including:
(i) receive a plurality of time frames including a first engagement period and a second engagement period from a distinct and remote controller generated via teacher input;
(ii) receive signals generated via teacher input from the remote controller, wherein the signals are based on student engagement observed by the teacher during the first engagement period, and the signals represent positive feedback and no signals are sent representing inappropriate behavior; and
(iii) notify the student by altering the display to represent a decline in student engagement in the event the teacher has not observed sufficient student engagement during the engagement period and has not generated the signal representing positive student engagement; and
wherein student engagement is independent and distinct from student behavior, and student engagement includes a degree of attention, curiosity, interest, optimism, and passion shown by a student when learning or being taught.

12. The wearable device of claim 11, wherein the student engagement consists of only the degree of attention, curiosity, interest, optimism, and passion shown by the student when learning or being taught and does not include student behavior.

13. The wearable device of claim 11, further comprising:
at least one light in the display that illuminates in response to signals generated from a remote controller, wherein the light remains illuminated during the engagement period and either darkens or changes color after the illumination period if no signal representing positive student engagement was received by the wearable device from the remote controller operated by the teacher.

14. The wearable device of claim 13, further comprising:
wherein the at least one light is one of three light emitting diodes (LEDs),
wherein the engagement period is one of three engagement periods, and
wherein each one of the LEDs is associated with one engagement period.

15. The wearable device of claim 14, further comprising:
a receiver carried by the housing to receive signals from the remote controller, wherein if the receiver receives a positive student engagement signal from the controller during a first engagement period, then all three LEDs remain illuminated in a first color, and if the receiver does not receive the positive student engagement signal from the controller during the first engagement period, then one of the LEDs illuminates in a second color until the receiver receives the positive student engagement signal.

16. The wearable device of claim 15, wherein each one of the three engagement periods is in a range from five minutes to fifteen minutes.

17. The wearable device of claim 15, further comprising:
communication logic electrically connected with the at least one non-transitory computer readable storage medium effectuating wireless data transfer between the wearable device and a central server hosting an application to display results of student engagement to a third party.

18. The wearable device of claim 17, further comprising:
timing logic in operative communication with the at least one non-transitory computer readable storage medium having instructions encoded thereon configured to initiate a countdown timer during which the student is to maintain his or her student engagement.

19. A student engagement system comprising:
a plurality of wearable electronic devices including wireless communication logic, wherein one student wears one wearable electronic device;
a remote control operated by the teacher, wherein the remote control includes teacher-selected input cues configured to identify student engagement;
one or more computing devices hosting a student portal associated with one of the plurality of students, wherein the computing device includes wireless communication logic;
a wireless network linking the plurality of wearable electronic devices, the remote control and the one or more computing devices;
wherein the teacher enters and sends signals generated by subjective feedback of student engagement from the remote to the wearable electronic device to each student his or her level of engagement to foster student learning; and
wherein student engagement is independent and distinct from student behavior, and student engagement includes a degree of attention, curiosity, interest, optimism, and passion shown by a student when learning or being taught.
